SEQUENCE LISTING (17 sequences)

SEQ ID NO: 1 - DNA, 3804 bp, Corynebacterium glutamicum
  CDS (1538)..(3289)
agagcgcacctaatggctgctgattctgatctcagtgttcacgatgcttacttaaaggagcatgttgcacctgtaaaggcgatcaactggaactccatcccagattccaaagatcttgaagtctgggatcgtctgaccggtaacttctggctcccagaaaaggtcccagtatccaacgacatcaagagctggggaaccctcaacgaggttgaaaaagccgcaaccatgcgcgtgttcaccggacttaccctgctggacaccattcagggcactgtcggcgcaatctccctgcttccagacgcagattcactgcacgaagaagcggtgctaaccaacattgcgttcatggaatccgtgcacgcaaagagttactccaacatcttcatgactctggcctccaccgcggaaatcaacgatgcgttccgttggtctgaggaaaatgaaaacctgcagcgcaaggcaaagatcatcctgtcttactatgagggcgatgatccactaaagcgcaagatcgcctccgtgatcctggagtccttcctgttctactccggcttctacctcccaatgtattggtccagccactccaagctgaccaacaccgccgacgtgatccgcctgatcatccgcgatgaggcagtgcacggctactacattggctacaagtatcaaaaggctgtcgcgaaggagactccagagcgtcaggaagagctgaaggagtacaccttcgatctgctctacgatctttacgataacgaaactcagtactccgaagatctctacgacgatcttggatggaccgaggatgttaagcgattccttcgctacaacgccaacaaggccctcaacaaccttggctacgaaggactcttcccagcggatgaaaccaaggtgtccccaaacatcttgtctgcgctgtcaccaaacgctgatgagaaccacgacttcttctccggctccggttcctcttacgttattggtaaggcagaaaacaccgaggatgatgactgggacttctaacttttaaaaagctgaagcgctctacggctgtagttaactgcaaccgttagagcgtttttcgctttctggtgggggcttaaggtgcgggtttttccgaagcgcatatcggggtaggggagcgccaggcgccccgtggacccctcggcggcacatcacgctttagaagaaaacgcccctggaatgggcgtctcaaccattcgattgaaccccggcggggggaattgtgaaatctgtggcaggggttaaccgtgggggtagggcttcctggcgaaatgtccgtcaaattgtgaaccccttcacacctttggttaaaagtcactgcccacaagtgactgaacctggcagcgacctcatgaattgtttgaaaaacattttttttgggcatgaaaaggggatacagttagctgcataccggcctttttgggttggcatcggatcctgcctgtggcctaagatcaggcagtgttgttaaaggacgatcggtaatccgaatggatcgtcccgtagtcaggaggaacctatgaccgctgtggcgcctagggtcgacgggcacgtcgcccctcagaggcccgagccgacaggccatgcacgcaagggcagcaaagcatggttaatgatgaccaccaccgaccacaagcagctgggcattatgtacatcattatgtccttcagcttcttcttcctcggtggcttgatggccctgcttatccgagcggagcttttcacccctggtctgcagttcctgtctaatgagcagttcaaccagctgttcaccatgcacggaactgtcatgctgctgctgtacggaactccaattgtttggggttttgctaactacgtcctgccacttcagatcggtgcgcctgacgtagctttcccacgtttgaatgctttcggcttctggatcaccaccgtcggtggtgtcgcgatgctgaccggcttcctgaccccgggtggtgctgccgacttcggttggaccatgtactccccactgtctgacgcaattcactccccaggccttggctctgacatgtggattgtcggtgtcggtgcaactggtattggctccgttgcttccgcaattaacatgctcaccaccatcctctgcctccgcgcacctggtatgaccatgttccgtatgcctattttcacctggaatatcttcgttgtttccgttcttgctctgctgatcttcccactgctgctcgctgctgcactgggtgttctgtatgaccgcaagcttggtggacacctgtacgatccagctaacggcggctccctcctgtggcagcacctgttctggttcttcggacaccctgaggtttacgttctggcgctgccgttcttcggcattgtttctgagatcattcctgtgttctcccgtaagccaatgttcggttacgtcggcctgatcttcgcaaccttgtccattggtgcactgtccatggctgtgtgggctcaccacatgttcgttactggcgcagttttgcttccgttcttctccttcatgacgttcctgatttcggttcctaccggcgttaagttcttcaactgggttggaaccatgtggaagggtcacatcacttgggaaaccccaatgatctggtctgttggcttcatggctaccttcctcttcggtggtctgaccggcattatgctggcgtccccaccactggacttccacttggctgactcctacttcctgatcgcgcacttccactacaccctcttcggtaccgtggtgttcgcatcgtgtgcaggcgtttacttctggttcccgaagatgactggccgcatgatggacgagcgtcttggcaagatccacttctggttgaccttcgtcggtttccacggaaccttcctcatccagcactgggtgggcaacatgggtatgccacgtcgttacgctgactacctggattctgatggtttcaccatctacaaccagatctccaccgtgttctccttcctgcttggcctgtctgtcattccattcatctggaacgtcttcaagtcctggcgctacggtgagctcgttaccgttgatgatccttggggttacggcaactccctggagtgggcaacctcctgccctcctcctcgccacaacttcgcatccttgcctcgtatccgctccgagcgccctgcgttcgagctgcactacccgcacatgattgaaagcatgcgcgcagaggcacacactggacatcacgatgatattaatgctccagaattgggtaccgccccagcccttgcatctgactccagccgctaaaagcgtctgatttaagtcggtacctgactaaataagcaccagccccagcagagataatctgccggggctggcgttttcatattccgacttggggcaccctgaatacatctcacccaattccccataactagacaattgcccagcaacgactgataagtctccaatgtcgtgttccgcgctcagacatgagacaattgttgccgtgactgaactcatccagaatgaatcccaagaaatcgctgagctggaagccggccagcaggttgcattgcgtgaaggttatcttcctgcggtgatcacagtgagcggtaaagaccgcccaggtgtgactgccgcgttctttagggtcttgtccgctaatcaggttcaggtcttggacgttgagcagtcaatgttccgtggctttttgaacttggcggcgtttgtgggtatcgcacctgagcgtgtcgagaccgtcaccacaggcctgactgacaccctcaaggtgcatggacagtccgtggtggtggagc

SEQ ID NO: 2 - protein, 584 aa, Corynebacterium glutamicum
MTAVAPRVDGHVAPQRPEPTGHARKGSKAWLMMTTTDHKQLGIMYIIMSFSFFFLGGLMALLIRAELFTPGLQFLSNEQFNQLFTMHGTVMLLLYGTPIVWGFANYVLPLQIGAPDVAFPRLNAFGFWITTVGGVAMLTGFLTPGGAADFGWTMYSPLSDAIHSPGLGSDMWIVGVGATGIGSVASAINMLTTILCLRAPGMTMFRMPIFTWNIFVVSVLALLIFPLLLAAALGVLYDRKLGGHLYDPANGGSLLWQHLFWFFGHPEVYVLALPFFGIVSEIIPVFSRKPMFGYVGLIFATLSIGALSMAVWAHHMFVTGAVLLPFFSFMTFLISVPTGVKFFNWVGTMWKGHITWETPMIWSVGFMATFLFGGLTGIMLASPPLDFHLADSYFLIAHFHYTLFGTVVFASCAGVYFWFPKMTGRMMDERLGKIHFWLTFVGFHGTFLIQHWVGNMGMPRRYADYLDSDGFTIYNQISTVFSFLLGLSVIPFIWNVFKSWRYGELVTVDDPWGYGNSLEWATSCPPPRHNFASLPRIRSERPAFELHYPHMIESMRAEAHTGHHDDINAPELGTAPALASDSSR

SEQ ID NO: 3 - DNA, 4112 bp, Corynebacterium glutamicum
  CDS (604)..(1680)
gcatgcatggcaaggcccgctcgagtgcaggaacgaatgcttcagcgttcccatttgcagtcaatatgccaagaaggccgcacatgattgaaaaatcctcctgaaataaaaggcgccttaaatcgcaaagaaattttgttggaagaaataagacgctgcatttgttaaatctcgtgtcaacgatacggcgaagttacatctgaggtgaaaagggcacgccaaaattgacgaaagctccctcaagcaacgtgcgtcagctgactattgcagcattctcaaaggttcctgaaaaccagattgatttcctaatacatgcaccttgtaggaacgtagggggtaagggtgggggaatttcaagggcaatcaaaaggttgatggtctgtgacgtggcatacaccaattgcctagactttaggtattccacctgaggattcgggcatatcgttgcagttgaaagacatttgacgcccctaaaaacgaaacccacgaagatatttccaccaaacacaagatatggaatcggctggcaaataggctattctgcgaagatagaaatgaccgtaaggtctctggtttttgtgtggacaggaaggcagaacacacgtggaacagcaaaataagcgtggtttaaagcgcaaggccctgcttggcggtgtcttgggcttaggtggcctcgccatggcaggctgtgaagtcgcccctcctggcggtgtgcttggagatttcctacgtatgggttggcctgatggcattacccctgaagcagtggccatgggtaacttctggtcatgggtctgggttgctgcctgcatcatcggcatcatcatgtggggtctattcctcaccgccatctttgcctggggcgcaaagagggctgaaaagcgcggcgagggtgaattccctaagcagctccagtacaacgttccacttgagctcgttctgacgatcgttccgatcatcattgttatggtgctgttcttcttcaccgttcaaactcaggacaaggtcaccgctctggataagaacccagaggttaccgtggacgtcaccgcttaccagtggaactggaagttcggatactccgaaattgatggctcactggcacctggtggacaggattaccaaggaagcgacccggagcgtcaggcagctgccgaggcttccaagaaggatccttctggagataacccaattcacggcaactcaaagtctgacgtttcttaccttgagttcaaccgaattgaaaccctcggaaccactgatgaaatcccagtgatggttcttcctgtgaacaccccaatcgagttcaacctcgcatctgctgacgttgcacactccttctgggttccagagttcctcttcaagcgagatgcttacgcacaccctgaggcaaacaagtcccagcgtgtcttccagattgaagagatcactgaggaaggcgcattcgttggtcgctgtgcagaaatgtgcggtacttaccacgcaatgatgaacttcgagcttcgtgtcgtcgatcgcgattccttcgctgtgtacatcagcttccgtgactccaacccagacgcaaccaacgctcaggcacttgagcacattggtcaagctccttacgctacttccactagcccattcgtttccgatcgcaccgcaacccgcgacggcgaaaacactcagagcaacgcttaagaaggagtggcgaaaaaatgaagtcttcagcaaaactcatgtacggcccgaccgtattcatggccgcaatggctgtcatctacatcttcgcaacaatgcacgttagtgatggcggcagcgttaaaggtgttgagtgggtcggttctgtggccctggtcctgtcagcaggtctgacgcttatgctcggtgtctaccttcacttcactgaagtccgcgtagatgttcttccagaggactgggaagaggctgaggttgccgacaaggcaggaaccctcgggttcttcagcccaagctccatttggccggcagctatgtccggtgcggttggattccttgcattcggcgttgtgtacttccactactggatgatcgcagttggtctgatgctcctgatcttcacgatcaccaagctcaaccttcagtacggcgtgccaaaagaaaagcactagtactaaaaccacatatgctcaaccctattggctgtccgatagggttgagcattttgtcgttttcacaggcttttccaactttgatcaccgtgacgaaactatcccaacagggcagaccgaggcgctgtgaaatacgaaaatcggcaaaattgaaaaagttcccgcaaaatcgagaaaagtttctgaaagcggaaaagtgggggagtgggcggacatgaatcgcattaagctgcaaaaacgccttttattcgggttcaaccaaaagttgtatgtggggctccaatggggtgaaaaactctgtgacgtgtctctcaggcaaaaagctgcggaaaccactttgggcacactctgagagttcttcaaaatgggacgagtgtgatttggggcagattggaactgcctgtgaaccctttaacctgcaattatgtaactgtcgtaaaagggctacgaaaagttcccggaaggtcgattgaaaagtttgcgaattgggggaaaattcgcatcaaaagccgagttcaaactttcaattgaaacggggggcttgaagtgactttggccaaccaaacagccatactagatagcgtgacgagcgcagttggaaatacaggtatggcagcaccacaacgtgttgcggcactgaaccgtccgaatatggtcagtgtcggcaccattgtgttcctgtctcaggaattaatgttcttcgccgggctattcgcgatgtacttcgtgtcccgtgcgaacggactggcaaatggatcatggggagagcagacagatcacctcaacgtgccctacgcactgttgattacggtcattctggtgtcttcctcagtgacttgccagttcggagtttttgcggctgaaaggggtgacgtttacggcctccgcaagtggttcttggtcacgattatcctcggatcaatcttcgtgatcggacagggctacgagtacatcactctcgtaggtcacggacttacaatccagagcagtgtctacggatcggcattctttattacaaccggtttccacgcactgcacgtgatcgcgggtgttatggccttcgttgtggttcttatgagaatccataagtcgaagttcactccggcacaggcaaccgcagcaatggttgtgtcttattactggcacttcgttgacgtggtctggatcggcctcttcatcactatttacttcattcagtaggcagtaaggaatcctcaacgttgttgaggttccctatgcccttcacttccacagtcgagattcaaagggaaatgatggaaaccaacccgcagaccccagagggaaatagcatggctaaaccctctgctaagaaggtcaagaatcgccgcaaggtccggcgcaccgtcgcaggtgcattggctctgaccattggactgagcggagcaggaatcctcgcaaccgcgatcactccagatgctcaagttgctaccgctcagcgtgacgatcaggcacttatctccgagggtaaagacctctacgatgtcgcctgcatcacctgccacggcgtaaacctccaaggtgttgaggaccgcggtccttccctcgtaggtgttggcgaaggcgcagtgtacttccaagttcactccggccgtatgccaatactgcgtaacgaggctcaggctgagcgcaaggctcctcgttacaccgaggcacagacccttgcgatcgctgcatatgttgcagctaatggcggtggcccaggactcgtttacaacgaggacggcaccctcgccatggaggagctccgtggcgaaaactacgacggacagattacctccgccgacgtcgctcgcggcggagatctgttccgcctgaactgtgcatcctgccacaacttcactggtcgtggtggcgcactgtcctctggtaagtacgcaccaaacctggatgctgcaaacgagcaggaaatctaccaggctatgcttaccggtcctcagaacatgcctaagttctccgatc

SEQ ID NO: 4 - protein, 359 aa, Corynebacterium glutamicum
VEQQNKRGLKRKALLGGVLGLGGLAMAGCEVAPPGGVLGDFLRMGWPDGITPEAVAMGNFWSWVWVAACIIGIIMWGLFLTAIFAWGAKRAEKRGEGEFPKQLQYNVPLELVLTIVPIIIVMVLFFFTVQTQDKVTALDKNPEVTVDVTAYQWNWKFGYSEIDGSLAPGGQDYQGSDPERQAAAEASKKDPSGDNPIHGNSKSDVSYLEFNRIETLGTTDEIPVMVLPVNTPIEFNLASADVAHSFWVPEFLFKRDAYAHPEANKSQRVFQIEEITEEGAFVGRCAEMCGTYHAMMNFELRVVDRDSFAVYISFRDSNPDATNAQALEHIGQAPYATSTSPFVSDRTATRDGENTQSNA

SEQ ID NO: 5 - protein, 205 aa, Corynebacterium glutamicum
VTSAVGNTGMAAPQRVAALNRPNMVSVGTIVFLSQELMFFAGLFAMYFVSRANGLANGSWGEQTDHLNVPYALLITVILVSSSVTCQFGVFAAERGDVYGLRKWFLVTIILGSIFVIGQGYEYITLVGHGLTIQSSVYGSAFFITTGFHALHVIAGVMAFVVVLMRIHKSKFTPAQATAAMVVSYYWHFVDVVWIGLFITIYFIQ

SEQ ID NO: 6 - DNA, 4683 bp, Corynebacterium glutamicum
  CDS (276)..(1124)
cacgtgatcgcgggtgttatggccttcgttgtggttcttatgagaatccataagtcgaagttcactccggcacaggcaaccgcagcaatggttgtgtcttattactggcacttcgttgacgtggtctggatcggcctcttcatcactatttacttcattcagtaggcagtaaggaatcctcaacgttgttgaggttccctatgcccttcacttccacagtcgagattcaaagggaaatgatggaaaccaacccgcagaccccagaggaaaatagcatggctaaaccctctgctaagaaggtcaagaatcgccgcaaggtccggcgcaccgtcgcaggtgcattggctctgaccattggactgagcggagcaggaatcctcgcaaccgcgatcactccagatgctcaagttgctaccgctcagcgtgacgatcaggcacttatctccgagggtaaagacctctacgctgtcgcctgcatcacctgtcacggcgtaaacctccaaggtgttgaggaccgcggtccttccctcgtaggtgttggcgaaggcgcagtgtacttccaagttcactccggccgtatgccaatgctgcgtaacgaggctcaggctgagcgcaaggctcctcgttacaccgaggcacagacccttgcgatcgctgcatatgttgcagctaatggcggtggcccaggactcgtttacaacgaggacggcaccctggccatggaggagctccgtggcgaaaactacgacggacagattacctccgccgacgtcgctcgcggcggagatctgttccgcctgaactgtgcatcctgccacaacttcactggtcgtggtggcgcactgtcctctggtaagtacgcaccaaacctggatgctgcaaacgagcaggaaatctaccaggctatgcttaccggtcctcagaacatgcctaagttctccgatcgtcagctctccgcagatgagaagaaggacatcatcgccttcatcaagtccaccaaggagactccatcacctggtggttactcactcggtagcttgggcccagtggctgagggtctgttcatgtgggtattcggcatcttggtcctcgtggccgccgctatgtggattggatcacgttcatgagtaacaacaacgacaagcagtacacaacccaagaactcaacgcgatgagcaatgaggatcttgcacgacttggtacagagctggacgacgttaccattgcataccgcaaggaacgtttcccaatccctaatgacccagctgagaagcgcgctgcacgttcagttactttctggctagtcctcggcatcattggtggacttggattcctggctacctacattttctggccttgggagtacaaggcacacggagatgaaggtctcctggcgtacaccttgtacaccccaatgctgggtattacttccggtctttgcatcctgtccctgggatttgcagttgtcctttatgtcaagaagttcattccagaggaaatcgcagtacagcgtcgccacgacggtccttctgaagaagttgaccgccgcaccatcgttgcacttctcaatgactcttggcagacctctactcttggtcgtcgcaagctgatcatgggacttgcaggtggcggagcagtactggccggcctgaccatcatcgctccaatgggcggtatgatcaagaatccttggaagcctaaggaaggcccaatggacgttcagggtgacggcaccctatggacttccggttggacactcattgagaacgacgtcaaggtttacctcggccgtgacactgcagcaattgcggagtcacacaccgatgcaaccggtgagcactggtcaaccactggtgtttcccgcctggttcgtatgcgcccagaagatctggcagcagcatccatggaaactgtcttcccacttccagctgaaatggtgaacgacggtgctgaatacgatcctgcgaaggacgtctacgagcaccaaatgcactcggtgcacggcccacgtaacgcagttatgttgatccgtctccgtaccgctgacgctgaaaaggttatcgaacgcgaaggccaggagtccttccactacggtgactactacgcttactccaagatttgtacacacattggttgtccaacctcactgtacgaggctcagaccaaccgtattctgtgcccatgtcaccagtcgcagtttgacgcattgcactacggaaagccagtcttcggacctgctgcccgtgcgctgccacagctgccaattaccgttgatgaagagggctacctcatcgccgctggtaacttcattgagccactcggccctgcattctgggagcgtaagtcatgagtctagctaccgtgggaaacaatcttgattcccgttacaccatggcgtcgggtatccgtcgccagatcaacaaggtcttcccaactcactggtccttcatgctcggcgagattgcgctttacagcttcatcgtcttgctgctgactggtgtctacctgaccctgttcttcgacccatcaatcaccaaggtcatttatgacggcggctacctcccactgaacggtgtggagatgtcccgtgcatacgcaactgcgttggatatttccttcgaagttcgcggtggtctgttcatccgccagatgcaccactgggcagccctgctgttcgttgtatccatgctggttcacatgctccgtattttcttcaccggtgcgttccgtcgcccacgtgaagcaaactggatcatcggtgttgttctgatcatcctgggtatggctgaaggcttcatgggttactccctgcctgatgacctgctctctggtgttggtcttcgaatcatgtccgccatcatcgttggtcttccgatcatcggtacctggatgcactggctgatcttcggtggagacttcccatccgatctgatgctggaccgcttctacatcgcacacgttctaatcatcccagctatcctgcttggcttgatcgcagctcacctggcacttgtttggtaccagaagcacacccagttcccaggcgctggccgcactgagaacaacgtggtcggtatccgaatcatgcctctgttcgcagttaaggctgttgctttcggcctcatcgtcttcggcttcctcgcactgcttgctggtgtcaccaccattaacgcaatttggaatcttggaccgtacaacccttcacaggtgtctgctggttcccagcctgacgtttacatgctgtggacagatggtgctgctcgtgtcatgccggcatgggagctctacctcggtaactacaccatcccagcagtcttctgggttgctgtgatgctgggtatcctcgtggttctgcttgtgacttacccattcattgagcgtaagttcactggcgacgatgcacaccacaacttgctgcagcgtcctcgcgatgttccagtccgcacctcactcggtgtcatggcgcttgtcttctacatcctgcttaccgtttctggtggtaacgatgtttacgcaatgcagttccatgtttcactgaacgcgatgacctggatcggtcgtatcggcctcatcgttggaccagctattgcatacttcatcacttaccgactgtgcatcggcttgcagcgctctgaccgcgaggtcctggagcacggcatcgagaccggtatcatcaagcagatgccaaatggtgccttcattgaagttcaccagccacttggcccagttgatgaccatggtcacccaatcccactgccatacgctggcgctgcggttccaaagcagatgaaccagcttggttacgctgaggttgaaacccgcggtggattcttcggacctgatccagaagacatccgtgcgaaggctaaggaaattgagcacgcaaaccacattgaggaagcgaacactcttcgtgcactcaacgaggcaaacattgagcgtgacaagaatgagggcaagaactagtttctaggacttcatctctgaaactccccgctgtagggacctgaatcgaaaggtctccgcatcggggagtttttctctattcagacgaggctaaagataagggagagggctctttaacacacgaggagtggcgtagagcctgtagttgccttatatgtagcttgtggcggcgtgaagcaacgtgcaggcgcgtggaaagcgtagagttttcttctccttatatataaggagtgttcttcgtcgtgagcattacgccctaggtcgcctggggtcggtgtccaacttcgctcaaattccgctcaaaatccagctcggtgtggcttaagaattgttgtcataactccaaatctcaaagcataagccgtcaatggtgattaatgtcacatggtgagatcattgctgaaactggtgccgatttttcggctctgtgaaaacgatttgactactggaagtttcctgaaattgcaggtcatctagctttctcagggttctaggggagaacccttagtggttggggtctgagtggaggacttgcgtctcggtcaaattaatccgcgataacggttcgataacgaccaattttttcgcttgggctagacaagtgttgttgcggtttcgtaaccttattgagacattgcgggacggacaccgaatttccgccagcattacagaaacaaatagacgcttaatcgcaagcatagtttagagaaattcttt

SEQ ID NO: 7 - protein, 283 aa, Corynebacterium glutamicum
MAKPSAKKVKNRRKVRRTVAGALALTIGLSGAGILATAITPDAQVATAQRDDQALISEGKDLYAVACITCHGVNLQGVEDRGPSLVGVGEGAVYFQVHSGRMPMLRNEAQAERKAPRYTEAQTLAIAAYVAANGGGPGLVYNEDGTLAMEELRGENYDGQITSADVARGGDLFRLNCASCHNFTGRGGALSSGKYAPNLDAANEQEIYQAMLTGPQNMPKFSDRQLSADEKKDIIAFIKSTKETPSPGGYSLGSLGPVAEGLFMWVFGILVLVAAAMWIGSRS

SEQ ID NO: 8 - protein, 392 aa, Corynebacterium glutamicum
MSNEDLARLGTELDDVTIAYRKERFPIPNDPAEKRAARSVTFWLVLGIIGGLGFLATYIFWPWEYKAHGDEGLLAYTLYTPMLGITSGLCILSLGFAVVLYVKKFIPEEIAVQRRHDGPSEEVDRRTIVALLNDSWQTSTLGRRKLIMGLAGGGAVLAGLTIIAPMGGMIKNPWKPKEGPMDVQGDGTLWTSGWTLIENDVKVYLGRDTAAIAESHTDATGEHWSTTGVSRLVRMRPEDLAAASMETVFPLPAEMVNDGAEYDPAKDVYEHQMHSVHGPRNAVMLIRLRTADAEKVIEREGQESFHYGDYYAYSKICTHIGCPTSLYEAQTNRILCPCHQSQFDALHYGKPVFGPAARALPQLPITVDEEGYLIAAGNFIEPLGPAFWERKS

SEQ ID NO: 9 - DNA, 4683 bp, Corynebacterium glutamicum; identical to SEQ ID NO: 6
  CDS (2347)..(3963)

SEQ ID NO: 10 - protein, 539 aa, Corynebacterium glutamicum
MSLATVGNNLDSRYTMASGIRRQINKVFPTHWSFMLGEIALYSFIVLLLTGVYLTLFFDPSITKVIYDGGYLPLNGVEMSRAYATALDISFEVRGGLFIRQMHHWAALLFVVSMLVHMLRIFFTGAFRRPREANWIIGVVLIILGMAEGFMGYSLPDDLLSGVGLRIMSAIIVGLPIIGTWMHWLIFGGDFPSDLMLDRFYIAHVLIIPAILLGLIAAHLALVWYQKHTQFPGAGRTENNVVGIRIMPLFAVKAVAFGLIVFGFLALLAGVTTINAIWNLGPYNPSQVSAGSQPDVYMLWTDGAARVMPAWELYLGNYTIPAVFWVAVMLGILVVLLVTYPFIERKFTGDDAHHNLLQRPRDVPVRTSLGVMALVFYILLTVSGGNDVYAMQFHVSLNAMTWIGRIGLIVGPAIAYFITYRLCIGLQRSDREVLEHGIETGIIKQMPNGAFIEVHQPLGPVDDHGHPIPLPYAGAAVPKQMNQLGYAEVETRGGFFGPDPEDIRAKAKEIEHANHIEEANTLRALNEANIERDKNEGKN

SEQ ID NO: 11 - DNA, 20 bp, ARTIFICIAL SEQUENCE
  SYNTHETIC DNA
tcatggtntgggyncaycay

SEQ ID NO: 12 - DNA, 20 bp, ARTIFICIAL SEQUENCE
  SYNTHETIC DNA
ataacrtwrtgraartgngc

SEQ ID NO: 13 - protein, 21 aa, Corynebacterium glutamicum
EVAPPGGVLGDFLRMGWPDGI

SEQ ID NO: 14 - DNA, 20 bp, ARTIFICIAL SEQUENCE
  SYNTHETIC DNA
ggygayttcytbcgnatggg

SEQ ID NO: 15 - DNA, 20 bp, ARTIFICIAL SEQUENCE
  SYNTHETIC DNA
ggaccgcasarytcngmrca

SEQ ID NO: 16 - protein, 16 aa, Corynebacterium glutamicum
QAERKAPRITEAQVLA

SEQ ID NO: 17 - protein, 24 aa, Corynebacterium glutamicum
LRGENWDGQITSADVARGGDLFRL

(19) United States
(12) Patent Application Publication
(10) Pub. No.: US 2002/0106669 A1
(43) Pub. Date: Aug. 8, 2002

(54) Respiratory chain enzyme genes of coryneform bacteria

(75) Inventor: Nobuhito Sone, Iizuka (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: Ajinomoto Co., Inc., Chuo-ku (JP)

(21) Appl. No.: 09/945,825

(22) Filed: Sep. 5, 2001

(30) Foreign Application Priority Data
Sep. 6, 2000 (JP) 2000-270283

(51) Int. Cl. C12Q 1/68; C07H 21/04; C12N 9/02; C12N 1/21; C12P 21/02

(52) U.S. Cl. 435/6; 435/189; 435/252.3; 536/23.2; 435/320.1; 435/69.1

(57) ABSTRACT

A gene coding for aa3 subunit I is obtained from a Corynebacterium glutamicum chromosomal DNA library by hybridization utilizing a probe produced based on a sequence common to a wide range of heme-copper enzymes. A gene coding for subunit II and a gene coding for subunit III are obtained by hybridization using a probe produced by using primers prepared based on the N-terminus amino acid sequence of subunit II, which is determined by using purified cytochrome aa3, and an operon coding for cytochrome bc1 complex existing downstream from the foregoing genes is further obtained.

### BACKGROUND OF THE INVENTION

1. Field of the Invention

The present invention relates to respiratory chain enzymes of coryneform bacteria, more precisely enzymes constituting the electron transport system, and genes coding for the enzymes.

2. Description of the Related Art

Most of organisms acquire energy necessary for life activity by respiration. In higher organisms, carbohydrates, proteins, and aliphatic acids are degraded into acetyl-CoA by the glycolytic pathway and the β-oxidation in cytoplasm, and acetyl-CoA is degraded by the citric acid cycle in mitochondria. The resulting energy is saved as reducing power of NADH and FADH₂. Finally, NADH is completely oxidized to water by the subsequent electron transport system that is present on mitochondrial inner membranes, and a proton concentration gradient is formed in a coupled manner to the oxidation, and serves as driving force of the ATP synthesis.

Since the bacterial respiratory chain generally comprises various functional enzyme complexes depending on species and growing circumstance, the energy conservation efficiency may vary to a great extent. For example, Escherichia coli contains at least two kinds of quinol oxidases, bo type and bd type, which function as terminal oxidases in the respiratory chain. When a wild-type strain carrying the enzymes of the both types, a mutant strain carrying only the bo type, and a mutant strain carrying only the bd type are compared as for growth yield observed in aerobic culture, the growth yield is the lowest in the mutant carrying only the bd type enzyme, and depends on the kind of the terminal oxidases and their energy conservation efficiency (Lecture Abstract for The Conference of The Society for Bioscience and Bioengineering, Japan, 1995, Subject No. 357).

Coryneform bacteria such as Brevibacterium lactofermentum and Brevibacterium flavum are gram-positive and aerobic bacteria that are industrially utilized for amino acid producers. Although terminal oxidases of the respiratory chain have been well investigated as for those of Proteobacteria, which is phylogenetically quite far from the coryneform bacteria, and those of Bacillus subtilis and the thermophilic Bacillus, which are also gram-positive bacteria like the coryneform bacteria but phylogenetically somewhat different from them, the electron transport system of respiratory chain in coryneform bacteria has not been investigated in detail. It is considered that it is important to elucidate the electron transport system of the respiratory chain, which is the key of the energy metabolism, in coryneform bacteria in view of collecting fundamental data for improving productivity of useful substances. Further, if enzymes involved in the electron transport system of the respiratory chain in coryneform bacteria and genes therefor are identified, they may be useful for, for example, creating strains with higher energy efficiency.

Thus far, it has been reported that the respiration of Brevibacterium lactofermentum is coupled to the proton transport, and it involves cytochromes a, b and c (Kawahara, Y., et al., Agric. Biol. Chem., 52 (8), 1979-1983 (1988)), and further a gene coding for a cytochrome bd type quinol oxidase has been isolated (Japanese Patent Laid-open Publication (Kokai) No. 11-346776, Europe Patent Laid-open Publication 0 967 282(A2)).

Further, in Corynebacterium glutamicum, there is a cytochrome bc1 complex, and presence of at least two kinds of terminal oxidases, SoxM type oxidase and cytochrome bd type oxidase, is confirmed (The Second Symposium Concerning Metabolic Engineering, Lecture Abstracts, 1999). This shows that the electron transfer pathway form quinone pool to oxygen molecule include two kinds of pathways, a pathway utilizing cytochrome bc1 complex and SoxM type oxidase (Castresana J, Saraste M., Trends in Biochem. Sci., 20, 443-448 (1995)) and a pathway utilizing only the cytochrome bd type oxidase. It is considered that the former is an electron transfer pathway of high energy efficiency, in which proton translocation value for transfer of one electron is high, and the latter is an electron transfer pathway of low energy efficiency, in which proton translocation value for transfer of one electron is low.

By the way, a cytochrome aa3 type oxidase is structurally defined as a heme-copper oxidase, and classified into SoxM type oxidases. It is known that there are two kinds of SoxM type oxidases, i.e., cytochrome c oxidase and quinol oxidase, and they have high proton transportation ability. Oxidases showing homology to the cytochrome aa3 type oxidase already discovered in microorganisms are known for Paracoccus denitrificans, Bradyrhizobium japonicum, Rhodobacter sphaeroides, Synechococcus vulcanus, Thermus thermophilus, Bacillus subtilis, Bacillus stearothermophilus and so forth (Trumpower, B. L. and Gennis, R. B., Annu. Rev. Biochem., 63, 675-716 (1994); Cao, J. et al., J. Biol. Chem., 267, 24273-24278 (1992); Sone, N. et al., Biochim. Biophys. Acta., 1183, 130-138 (1993); Sakamoto, J. et al., J. Biochem., 122, 764-771 (1997)).

On the other hand, the cytochrome bc1 complexes widely exist in respiratory chains of various organisms, such as those in mitochondria and microorganisms, and constitute a superfamily. These enzymes play an important role in the production of energy required for organisms, that is, they transport electrons from quinol to cytochrome c, and simultaneously, pump out protons form the inside of membrane to the outside of membrane to form a transmembrane proton concentration gradient. Further, these cytochrome c1 reductases are considered to transport protons form the inside of membrane to the outside of membrane by a mechanism called proton motive Q cycle (Mitchell, P., J. Theoret. Biol., 62, 327-367 (1976); Croft, A. R., Meinhardt, S. W., Jones, K. R., and Snozzi, M., Biochim. Biophys. Acta, 723, 202-218 (1983); Trumpower, B. L., J. Biol. Chem., 265, 11409-11412 (1990)). The cytochrome bc1 complexes are known for Paracoccus denitrificans, Rhodobacter sphaeroides and Bacillus stearothermophilus (Trumpower B. L. and Gennis R B., Annu. Rev. Biochem., 63, 675-716 (1994), Sone et al., J. Biol. Chem., 271, 12457-12462 (1996)).

### SUMMARY OF THE INVENTION

An object of the present invention is to provide a cytochrome aa3 type oxidase and cytochrome bc1 complex of coryneform bacteria, as well as genes coding for them.

In order to achieve the aforementioned object, the inventors of the present invention attempted to clone a cytochrome aa3 gene of Corynebacterium glutamicum. As a result, they successfully cloned a gene coding for the aa3 subunit I (also referred to as “ctaD” hereinafter) from a chromosomal DNA library of Corynebacterium glutamicum by hybridization utilizing a probe produced based on a sequence common to a wide range of heme-copper oxidases. However, unlike many other oxidases, genes of other subunits were not found in the neighborhood of that gene.

On the other hand, the inventors of the present invention also successfully purified cytochrome aa3 from a membrane preparation of Corynebacterium glutamicum, and they isolated a subunit II protein from that purified enzyme, and determined its N-terminus partial peptide sequence. A primer was prepared based on this sequence, and PCR was performed by using this primer and a primer prepared based on the conserved sequence of CuA binding motif in the oxidase subunit II to produce a probe. Then, hybridization was performed by using this probe to successfully obtain a gene coding for the subunit II (also referred to as “ctac” hereinafter). Furthermore, it was also found that a gene coding for the subunit III (also referred to as “ctaE” hereinafter) existed downstream from that gene.

Further, a sequence estimated to be a part of ORF was found at a position on the 3′ end side and downstream from the aforementioned gene coding for the cytochrome aa3 subunit III (ctaE). Then, a clone containing this sequence was obtained and its sequence was determined. Furthermore, a subunit containing cytochrome c among the subunits constituting the cytochrome bc1 complex was purified, and its amino acid sequence was determined. As a result, it was found that the aforementioned clone contained an operon coding for the three subunits of cytochrome bc1 complex (qcrCAB).

The present invention was accomplished as described above, and provides the followings.

(1) A polypeptide defined in the following (A1) or (A2):

(A1) a polypeptide that has the amino acid sequence of SEQ ID NO: 2,

(A2) a polypeptide that has the amino acid sequence of SEQ ID NO: 2 comprising substitution, deletion, insertion or addition of one or several amino acid residues in the amino acid sequence, and can constitute cytochrome aa3 together with a cytochrome aa3 subunit II having the amino acid sequence of SEQ ID NO: 4 and cytochrome aa3 subunit III having the amino acid sequence of SEQ ID NO: 5.

(2) A polypeptide defined in the following (B1) or (B2):

(B1) a polypeptide that has the amino acid sequence of SEQ ID NO: 4,

(B2) a polypeptide that has the amino acid sequence of SEQ ID NO: 4 comprising substitution, deletion, insertion or addition of one or several amino acid residues in the amino acid sequence, and can constitute cytochrome aa3 together with a cytochrome aa3 subunit I having the amino acid sequence of SEQ ID NO: 2 and cytochrome aa3 subunit III having the amino acid sequence of SEQ ID NO: 5.

(3) A polypeptide defined in the following (C1) or (C2):

(C1) a polypeptide that has the amino acid sequence of SEQ ID NO: 5,

(C2) a polypeptide that has the amino acid sequence of SEQ ID NO: 5 comprising substitution, deletion, insertion or addition of one or several amino acid residues in the amino acid sequence, and can constitute cytochrome aa3 together with a cytochrome aa3 subunit I having the amino acid sequence of SEQ ID NO: 2 and cytochrome aa3 subunit II having the amino acid sequence of SEQ ID NO: 4.

(4) A cytochrome aa3 consisting of the polypeptides according to (1), (2) and (3).

(5) A polypeptide defined in the following (D1) or (D2):

(D1) a polypeptide that has the amino acid sequence of SEQ ID NO: 7,

(D2) a polypeptide that has the amino acid sequence of SEQ ID NO: 7 comprising substitution, deletion, insertion or addition of one or several amino acid residues in the amino acid sequence, and can constitute a cytochrome bc1 complex together with QcrA having the amino acid sequence of SEQ ID NO: 8 and QcrB having the amino acid sequence of SEQ ID NO: 10.

(6) A polypeptide defined in the following (E1) or (E2):

(E1) a polypeptide that has the amino acid sequence of SEQ ID NO: 8,

(E2) a polypeptide that has the amino acid sequence of SEQ ID NO: 8 comprising substitution, deletion, insertion or addition of one or several amino acid residues in the amino acid sequence, and can constitute a cytochrome bc1 complex together with QcrC having the amino acid sequence of SEQ ID NO: 7 and QcrB having the amino acid sequence of SEQ ID NO: 10.

(7) A polypeptide defined in the following (F1) or (F2):

(F1) a polypeptide that has the amino acid sequence of SEQ ID NO: 10,

(F2) a polypeptide that has the amino acid sequence of SEQ ID NO: 10 comprising substitution, deletion, insertion or addition of one or several amino acid residues in the amino acid sequence, and can constitute a cytochrome bc1 complex together with QcrC having the amino acid sequence of SEQ ID NO: 7 and QcrA having the amino acid sequence of SEQ ID NO: 8.

(8) A cytochrome bc1 complex consisting of the polypeptides according to (5), (6) and (7).

(9) A DNA coding for a polypeptide defined in the following (A1) or (A2):

(A1) a polypeptide that has the amino acid sequence of SEQ ID NO: 2,

(A2) a polypeptide that has the amino acid sequence of SEQ ID NO: 2 comprising substitution, deletion, insertion or addition of one or several amino acid residues in the amino acid sequence, and can constitute cytochrome aa3 together with a cytochrome aa3 subunit II having the amino acid sequence of SEQ ID NO: 4 and cytochrome aa3 subunit III having the amino acid sequence of SEQ ID NO: 5.

(10) A DNA coding for a polypeptide defined in the following (B1) or (B2):

(B1) a polypeptide that has the amino acid sequence of SEQ ID NO: 4,

(B2) a polypeptide that has the amino acid sequence of SEQ ID NO: 4 comprising substitution, deletion, insertion or addition of one or several amino acid residues in the amino acid sequence, and can constitute cytochrome aa3 together with a cytochrome aa3 subunit I having the amino acid sequence of SEQ ID NO: 2 and cytochrome aa3 subunit III having the amino acid sequence of SEQ ID NO: 5.

(11) A DNA coding for a polypeptide defined in the following (C1) or (C2):

(C1) a polypeptide that has the amino acid sequence of SEQ ID NO: 5,

(C2) a polypeptide that has the amino acid sequence of SEQ ID NO: 5 comprising substitution, deletion, insertion or addition of one or several amino acid residues in the amino acid sequence, and can constitute cytochrome aa3 together with a cytochrome aa3 subunit I having the amino acid sequence of SEQ ID NO: 2 and cytochrome aa3 subunit II having the amino acid sequence of SEQ ID NO: 4.

(12) A DNA coding for a polypeptide defined in the following (D1) or (D2):

(D1) a polypeptide that has the amino acid sequence of SEQ ID NO: 7,

(D2) a polypeptide that has the amino acid sequence of SEQ ID NO: 7 comprising substitution, deletion, insertion or addition of one or several amino acid residues in the amino acid sequence, and can constitute a cytochrome bc1 complex together with QcrA having the amino acid sequence of SEQ ID NO: 8 and QcrB having the amino acid sequence of SEQ ID NO: 10.

(13) A DNA coding for a polypeptide defined in the following (E1) or (E2):

(E1) a polypeptide that has the amino acid sequence of SEQ ID NO: 8,

(E2) a polypeptide that has the amino acid sequence of SEQ ID NO: 8 comprising substitution, deletion, insertion or addition of one or several amino acid residues in the amino acid sequence, and can constitute a cytochrome bc1 complex together with QcrC having the amino acid sequence of SEQ ID NO: 7 and QcrB having the amino acid sequence of SEQ ID NO: 10.

(14) A DNA coding for a polypeptide defined in the following (F1) or (F2):

(F1) a polypeptide that has the amino acid sequence of SEQ ID NO: 10,

(F2) a polypeptide that has the amino acid sequence of SEQ ID NO: 10 comprising substitution, deletion, insertion or addition of one or several amino acid residues in the amino acid sequence, and can constitute a cytochrome bc1 complex together with QcrC having the amino acid sequence of SEQ ID NO: 7 and QcrA having the amino acid sequence of SEQ ID NO: 8.

In the present invention, the expression that “a polypeptide can constitute cytochrome aa3” means that the polypeptide has a property that it can form a protein complex that shows oxidation-reduction spectrum of heme a, CO binding reduction-type minus reduction-type difference spectrum of heme a3, and an activity for receiving an electron from cytochrome c and transmitting the electron to oxygen so as to reduce it to a water molecule, together with the other subunit peptides. Further, the expression that “a polypeptide can constitute cytochrome bc1 complex” means that the polypeptide has a property that it can form a protein complex that shows oxidation-reduction absorption spectra of heme b and heme c, and an activity for receiving an electron from a reduced type quinone compound (quinol) and transmitting the electron to cytochrome c or a terminal oxidase, together with the other subunit peptides.

According to the present invention, there are provided subunits constituting cytochrome aa3 and DNA coding for them. There are also provided subunits constituting cytochrome bc1 complex and DNA coding for them. These polypeptides and DNA are useful for elucidation of the electron transport system of coryneform bacteria. Further, the DNA of the present invention can be used for breeding of coryneform bacteria that produce useful substances with good energy efficiency.

### BRIEF EXPLANATION OF THE DRAWINGS

FIG. 1 shows structure of genes coding for the subunits of cytochrome aa3, and relationship between the structures and clones:

(A) structure of DNA fragment containing the ctaC gene coding for subunit II and the ctaE gene coding for the subunit III, and

(B) structure of DNA fragment containing ctaD coding for the subunit I.

FIG. 2 shows structures of genes coding for subunits of cytochrome bc1 complex (qcrC, qcrA, qcrB), and relationship between the structures and clones.

### DETAILED DESCRIPTION OF THE INVENTION

Hereafter, the present invention will be explained in detail.

<1> Cytochrome aa3, Subunits Thereof, and DNA Coding for Them

Among the DNAs coding for cytochrome aa3 of the present invention, the DNA coding for the subunit I can be obtained as follows. That is, primers are prepared from, for example, Corynebacterium glutamicum chromosomal DNA based an amino acid sequence of a region highly conserved in known heme-copper enzymes such as cytochrome c oxidases I of Bradyrhizobium japonicum, Acetobacter aceti, Synechococcus vulcanus and Bacillus stearothermophilus, and a probe is prepared by PCR using the primers and chromosomal DNA of Corynebacterium glutamicum as a template. Then, the intended DNA can be obtained from a Corynebacterium glutamicum chromosomal DNA library by hybridization using the probe obtained above.

The chromosomal DNA of Corynebacterium glutamicum can be prepared by, for example, the method of Saito and Miura (Biochem. Biophys. Acta., 72, 619, (1963)), or the method of K. S. Kirby (Biochem. J., 64, 405, (1956)). A chromosome DNA library can be obtained by partially digesting chromosomal DNA with a suitable restriction enzyme, ligating each of the obtained DNA fragments to a vector DNA autonomously replicable in Escherichia coli cell to prepare recombinant DNA, and introducing the DNA into Escherichia coli. The vector is not particularly limited, so long as it is a vector usually used for genetic cloning, and plasmid vectors such as pUC19, pUC18, pUC118 and pUC119, phage vectors such as lambda phage DNA and so forth can be used.

The primers used for the aforementioned PCR may be, for example, oligonucleotides having the nucleotide sequences of SEQ ID NO: 11 or SEQ ID NO: 12.

Screening of a chromosomal DNA library of Corynebacterium glutamicum utilizing a DNA fragment obtained in PCR as a probe can be performed by colony hybridization when plasmid vectors are used for the preparation of the library, or plaque hybridization when phage vectors are used for the preparation of the library. A hybridization positive clone can be confirmed if it contains the target gene coding for cytochrome aa3 subunit I (ctaD) by preparing DNA from the clone and determining its nucleotide sequence. It is also possible to preliminarily perform Southern analysis for a hybridization positive clone by using the aforementioned probe.

A nucleotide sequence of ctaD gene of Corynebacterium glutamicum KY9002 strain (ATCC13032) obtained in the working example to be mentioned later in such a manner as described above is shown in SEQ ID NO: 1. An expected coding region and amino acid sequence of protein encoded thereby are shown in SEQ ID NO: 1.

Further, genes coding for cytochrome aa3 subunits II and III (ctaC and ctaE) can be obtained as follows.

From a membrane preparation of Corynebacterium glutamicum, cytochrome aa3 is purified, and an N-terminus partial peptide sequence of each subunit is determined. A primer is prepared based on the sequence. By using this primer and a primer prepared based on a sequence highly conserved among the known oxidase subunits, for example, a conserved sequence of CuA binding motif in the subunit II, PCR is performed to produce a probe. Then, a DNA fragment containing the gene coding for the subunit II (ctaC) and the gene coding for the subunit III (ctaE) is obtained by hybridization using the probe.

The primers used for the aforementioned PCR may be, for example, oligonucleotides having the nucleotide sequences of SEQ ID NO: 14 or SEQ ID NO: 15.

A nucleotide sequence of a DNA fragment containing ctaC gene and ctaE gene of the Corynebacterium glutamicum KY9002 strain (ATCC13032), which was obtained in the working example to be mentioned later in the manner as described above, is shown in SEQ ID NO: 3. Expected coding regions and amino acid sequences of proteins encoded thereby are shown in SEQ ID NOS: 4 and 5. In the aforementioned DNA fragment, the ctac gene and the ctaE gene were separate from each other by about 1 kb, and another ORF existed between them.

The numbers of amino acid residues of the subunits I, II and III of cytochrome aa3 are estimated to be 584, 316 and 205 residues, respectively, and molecular weights are calculated to be 65.0, 39.5 and 22.4 kDa, respectively. As for the subunit II, it is considered that an N-terminal sequence from first residue to the glycine residue at the 28th position is excised and the cysteine residue at the 29th position is modified with a lipid in a matured polypeptide in view of the results of the amino acid sequence analysis and analogy with oxidases of other species (Bacillus subtilis and Escherichia coli: Santana, M. et al., J. Biol. Chem., 267, 10225-10231 (1992)).

In the amino acid sequence of the subunit II shown in SEQ ID NO: 4, the amino acid numbers 257-268 correspond to the conserved sequence of CuA binding motif.

In addition, the codons of the amino acid residues of the N-termini of the subunits II and III are GTG, and the corresponding amino acid residues are indicated as Val in Sequence Listing. However, they are actually Met, and it is considered that this is because GTG is recognized as an initiation methionine. Other examples of such a phenomenon have also been reported.

Analyses such as estimation of coding regions and operon structure can be performed by using GENETYX Homology Version 2.2.2 (Software Development Co., Ltd.). Homology analysis can be performed according to the method of Lipman and Peason (Science, 227, 1435-1441, 1985).

The nucleotide sequences of ctaD, ctaC and ctaE were elucidated by the present invention, and therefore these gene can be directly obtained by preparing primers based on the nucleotide sequence shown in SEQ ID NO: 1 or 3, and performing PCR utilizing them and Corynebacterium glutamicum chromosomal DNA as a template.

The DNA of the present invention may be one coding for either one of the aforementioned subunits I, II and III of cytochrome aa3, or one coding for two or three kinds of them. Cytochrome aa3 or a subunit thereof can be produced by introducing the DNA of the present invention into a suitable host cell, and culturing an obtained transformant to express the DNA. For example, the DNA coding for the subunit I may be a DNA having the nucleotide sequence consisting of nucleotide numbers 1538-3289 in the nucleotide sequence of SEQ ID NO: 1, the DNA coding for the subunit II may be a DNA having the nucleotide sequence consisting of nucleotide numbers 604-1680 in the nucleotide sequence of SEQ ID NO: 3, and the DNA coding for the subunit III may be a DNA having the nucleotide sequence consisting of nucleotide numbers 2715-3329 in the nucleotide sequence of SEQ ID NO: 3.

Produced cytochrome aa3 or subunits thereof can be collected and purified from culture by a method commonly used for the purification of proteins such as salting out, solvent precipitation, gel filtration chromatography and ion exchange chromatography.

The DNA of the present invention coding for the subunit I may be one coding for a polypeptide that has the amino acid sequence of SEQ ID NO: 2 comprising substitution, deletion, insertion or addition of one or several amino acid residues in the amino acid sequence, and can constitute cytochrome aa3 together with the subunit II and III.

The DNA of the present invention encoding the subunit II may be one coding for a polypeptide that has the amino acid sequence of SEQ ID NO: 4 comprising substitution, deletion, insertion or addition of one or several amino acid residues in the amino acid sequence, and can constitute cytochrome aa3 together with the subunit I and III.

The DNA of the present invention encoding the subunit III may be one coding for a polypeptide that has the amino acid sequence of SEQ ID NO: 5 comprising substitution, deletion, insertion or addition of one or several amino acid residues in the amino acid sequence, and can constitute cytochrome aa3 together with the subunit I and II.

Furthermore, a DNA coding for a cytochrome aa3 in which Subunit I, II or III, or two or three of them contain a mutation also falls within the scope of the DNA of the present invention.

The number of “several” amino acid residues is preferably 1-40, more preferably 1-10. Alternatively, the number is preferably such a number that the amino acid sequence should show homology of 80% or more, preferably 95% or more, to the amino acid sequence of SEQ ID NO: 2, 4 or 5.

Such a DNA coding for substantially the same protein as the subunit I, II or III as described above can be obtained by, for example, modifying each nucleotide sequence by, for example, the site-directed mutagenesis method so that the amino acid sequence should involve substitution, deletion, insertion or addition of one or more amino acid residues at a specified site. Such a DNA modified as described above may also be obtained by a conventionally known mutation treatment. The mutation treatment includes a method of treating DNA coding for the subunit I, II or III in vitro, for example, with hydroxylamine, and a method for treating a microorganism, for example, a bacterium belonging to the genus Escherichia, harboring a DNA coding for the subunit I and/or subunit II with ultraviolet irradiation or a mutating agent usually used for mutation treatment such as N-methyl-N′-nitro-N-nitrosoguanidine (NTG) and nitrous acid.

The substitution, deletion, insertion or addition of nucleotide as described above also includes a naturally occurring mutation (mutant or variant) on the basis of, for example, individual difference or difference in species or genus of coryneform bacteria that harbor cytochrome aa3 and so forth.

A DNA coding for substantially the same protein as the subunit I, II or III described above can be obtained by expressing such a DNA having a mutation as described above in a suitable cell, and examining the cytochrome aa3 activity of the expression product. A DNA coding for substantially the same protein as the subunit I, II or III can also be obtained by isolating a DNA hybridizable with a DNA having, for example, the nucleotide sequence corresponding to nucleotide numbers of 1538-3289 of the nucleotide sequence shown in SEQ ID NO: 1, the nucleotide sequence corresponding to nucleotide numbers of 604-1680 or the nucleotide numbers of 2715-3329 of the nucleotide sequence shown in SEQ ID NO: 3 under the stringent conditions, and coding for a protein having a function of the subunit I, II or III from a DNA coding for the subunit I, II or III including a mutation or a cell harboring it. The “stringent conditions” referred to herein is a condition under which so-called specific hybrid is formed, and non-specific hybrid is not formed. It is difficult to clearly express this condition by using any numerical value. However, for example, the stringent conditions include a condition under which DNA's having high homology, for example, DNA's having homology of not less than 50%, are hybridized with each other, and DNA's having homology lower than the above level are not hybridized with each other. Alternatively, the stringent conditions are exemplified by a condition under which DNA's are hybridized with each other at a salt concentration corresponding to an ordinary condition of washing in Southern hybridization, i.e., 1× SSC, 0.1% SDS, preferably 0.1× SSC, 0.1% SDS, at 60° C.

Genes hybridizable under such conditions as described above include those having a stop codon generated in the genes, and those no longer having activity. However, such genes can be readily removed by ligating each of the genes with a commercially available activity expression vector, and examining the function of the expression product.

The host for the expression of the DNA of the present invention include, for example, various kinds of bacteria including coryneform bacteria such as Escherichia coli, Brevibacterium lactofermentum, and Brevibacterium flavum, eucaryotic cells such as Saccharomyces cerevisiae and so forth. In order to introduce the DNA of the present invention into a host such as those mentioned above, the host cell can be transformed with a recombinant vector which is obtained by inserting the DNA of the present invention into a vector selected depending on the kind of the host in which the expression is to be obtained. Those procedures can be performed by using methods of genetic recombination well known to those skilled in the art.

Methods for preparation of chromosomal DNA, construction of chromosomal DNA library, hybridization, PCR, preparation of plasmid DNA, digestion and ligation of DNA, transformation, design of oligonucleotides used as primers and so forth are described in by Sambrook, J., Fritsch, E. F., Maniatis, T., “Molecular Cloning A Laboratory Manual, Second Edition”, Cold Spring Harbor Laboratory Press (1989) and so forth.

The cytochrome aa3, subunits thereof and DNA coding for them of the present invention are considered to be useful for elucidation of the electron transport system of coryneform bacteria. Further, the DNA can be used for breeding of coryneform bacteria that produce useful substances with good energy efficiency.

<2> Cytochrome bc1 Complex, Subunits Thereof and DNA Coding for Them

A part of DNA coding for cytochrome bc1 complex was found in the DNA fragment containing the genes coding for cytochrome aa3 subunits II and III (ctaC and ctaE) obtained as described above, at a position downstream from the genes on the 3′ side. Therefore, a DNA coding for cytochrome bc1 complex can be obtained by cloning the flanking regions of the ctaE gene of Corynebacterium glutamicum chromosomal DNA in a known manner.

Specifically, PCR is performed (95° C. for 60 seconds, 52° C. for 60 seconds, 68° C. for 60 seconds, 35 cycles) by using the primers shown in SEQ ID NOS: 14 and 15 and chromosomal DNA of Corynebacterium glutamicum KY9002 strain (ATCC13032) as a template, and the obtained DNA fragment is used as a probe to screen a chromosomal DNA library by colony hybridization.

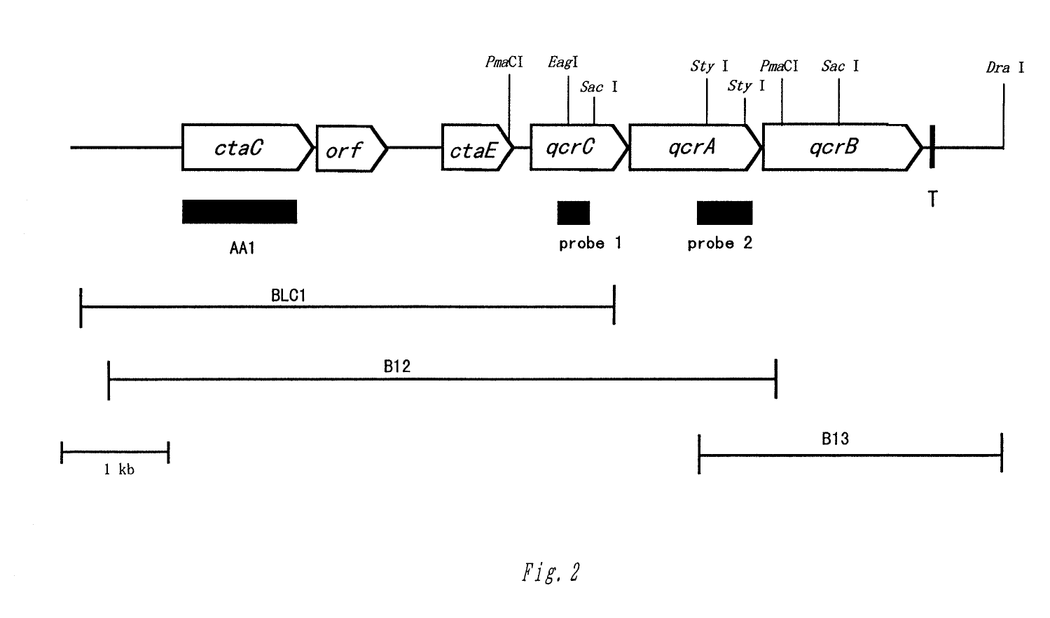

The nucleotide sequence of DNA coding for cytochrome bc1 complex of Corynebacterium glutamicum KY9002 strain (ATCC13032) obtained in the manner described above in the working example to be mentioned later is shown in SEQ ID NO: 6. This nucleotide sequence contains six ORFs, three of which (nucleotide numbers 276-1124, 1172-2347 and 2347-3963) are qcrC, qcrA and qcrB, respectively, and code for the subunits constituting the cytochrome bc1 complex (QcrC, QcrA, QcrB). These genes take an operon structure (FIG. 2). The amino acid sequences encoded by the ORFs are shown in SEQ ID NOS: 7, 8 and 10 in that order. In addition, the amino acid sequence encoded by qcrC and qcrA is shown in SEQ ID NO: 6 together with the nucleotide sequence. Further, the amino acid sequence encoded by qcrB is shown in SEQ ID NO: 9 together with the nucleotide sequence (the same as the nucleotide sequence shown in SEQ ID NO: 6).

The nucleotide sequences of qcrC, qcrA and qcrB were elucidated by the present invention, and therefore these genes can be directly obtained by preparing primers based on the nucleotide sequence shown in SEQ ID NO: 6, and performing PCR utilizing them and Corynebacterium glutamicum chromosomal DNA as a template.

The DNA of the present invention may be one coding for either one of the aforementioned subunits QcrC, QcrA and QcrB of the cytochrome bc1 complex, or one coding for two or three kinds of them. The cytochrome bc1 complex or a subunit thereof can be produced by introducing the DNA of the present invention into a suitable host cell, and culturing an obtained transformant to express the DNA. For example, the DNA coding for QcrC may be a DNA having the nucleotide sequence consisting of nucleotide numbers 276-1124 in the nucleotide sequence of SEQ ID NO: 6, the DNA coding for QcrA may be a DNA having the nucleotide sequence consisting of nucleotide numbers 1172-2347 in the nucleotide sequence of SEQ ID NO: 6, and the DNA coding for QcrB may be a DNA having the nucleotide sequence consisting of nucleotide numbers 2347-3963 in the nucleotide sequence of SEQ ID NO: 6.

Produced cytochrome bc1 complex or subunits thereof can be collected and purified from culture by a method commonly used for the purification of proteins such as salting out, solvent precipitation, gel filtration chromatography and ion exchange chromatography.

The DNA of the present invention coding for QcrC may be one coding for a polypeptide that has the amino acid sequence of SEQ ID NO: 7 comprising substitution, deletion, insertion or addition of one or several amino acid residues in the amino acid sequence, and can constitute the cytochrome bc1 complex together with QcrA and QcrB.

The DNA of the present invention coding for QcrA may be one coding for a polypeptide that has the amino acid sequence of SEQ ID NO: 8 comprising substitution, deletion, insertion or addition of one or several amino acid residues in the amino acid sequence, and can constitute the cytochrome bc1 complex together with QcrC and QcrB.

The DNA of the present invention coding for QcrB may be one coding for a polypeptide that has the amino acid sequence of SEQ ID NO: 10 comprising substitution, deletion, insertion or addition of one or several amino acid residues in the amino acid sequence, and can constitute the cytochrome bc1 complex together with QcrC and QcrA.

Furthermore, a DNA coding for a cytochrome bc1 complex in which QcrC, QcrA or QcrB, or two or three of them contain a mutation also falls within the scope of the DNA of the present invention.

The number of “several” amino acid residues is preferably 1-40, more preferably 1-10. Alternatively, the number is preferably such a number that the amino acid sequence should show homology of 80% or more, preferably 95% or more, to the amino acid sequence of SEQ ID NO: 7, 8 or 10.

Such a DNA coding for substantially the same protein as QcrC, QcrA or QcrB as described above can be obtained by the site-directed mutagenesis method or mutation treatment in the same manner as those mentioned above for DNA coding for substantially the same protein as cytochrome aa3.

A DNA coding for substantially the same protein as QcrC, QcrA or QcrB can also be obtained by isolating a DNA hybridizable with a DNA having, for example, the nucleotide sequence corresponding to nucleotide numbers of 276-1124, the nucleotide sequence corresponding to nucleotide numbers of 1172-2347 or the nucleotide numbers of 2347-3963 of the nucleotide sequence shown in SEQ ID NO: 6 under the stringent conditions, and coding for a protein having a function of QcrC, QcrA or QcrB from qcrC, qcrA or qcrB including a mutation or a cell harboring it. The “stringent conditions” has the same meaning as described above.

The host for the expression of the DNA of the present invention include, for example, various kinds of bacteria including coryneform bacteria such as Escherichia coli, Brevibacterium lactofermentum, and Brevibacterium flavum, eucaryotic cells such as Saccharomyces cerevisiae and so forth. In order to introduce the DNA of the present invention into a host such as those mentioned above, the host cell can be transformed with a recombinant vector which is obtained by inserting the DNA of the present invention into a vector selected depending on the kind of the host in which the expression is to be obtained. Those procedures can be performed by using methods of genetic recombination well known to those skilled in the art.

The cytochrome bc1 complex, subunits thereof and DNA coding for them of the present invention are considered to be useful for elucidation of the electron transport system of coryneform bacteria. Further, the DNA can be used for breeding of coryneform bacteria that produce useful substances with good energy efficiency.

### BEST MODE FOR CARRYING OUT THE INVENTION

The present invention will be more specifically explained with reference to the following examples.

### EXAMPLE 1

### Cloning of Cytochrome aa3 Subunit I Gene

(1) Cloning of Cytochrome aa3 Subunit I Gene of Corynebacterium glutamicum

Primers (uni1: TCATGGTNTGGGYNCAYCAY (SEQ ID NO: 11), uni2r: ATAACRTWRTGRAARTGNGC (SEQ ID NO: 12)) were synthesized based on the amino acid sequence of the region highly conserved in cytochrome c oxidase subunits I of Bradyrhizobium japonicum, Acetobacter aceti, Synechococcus vulcanus and Bacillus stearothermophilus. By using these primers and chromosomal DNA of Corynebacterium glutamicum KY9002 strain (ATCC13032) as a template, PCR was performed (95° C. for 45 seconds, 52° C. for 60 seconds and 62° C. for 90 seconds, 35 cycles) to obtain a fragment of 0.3 kb.

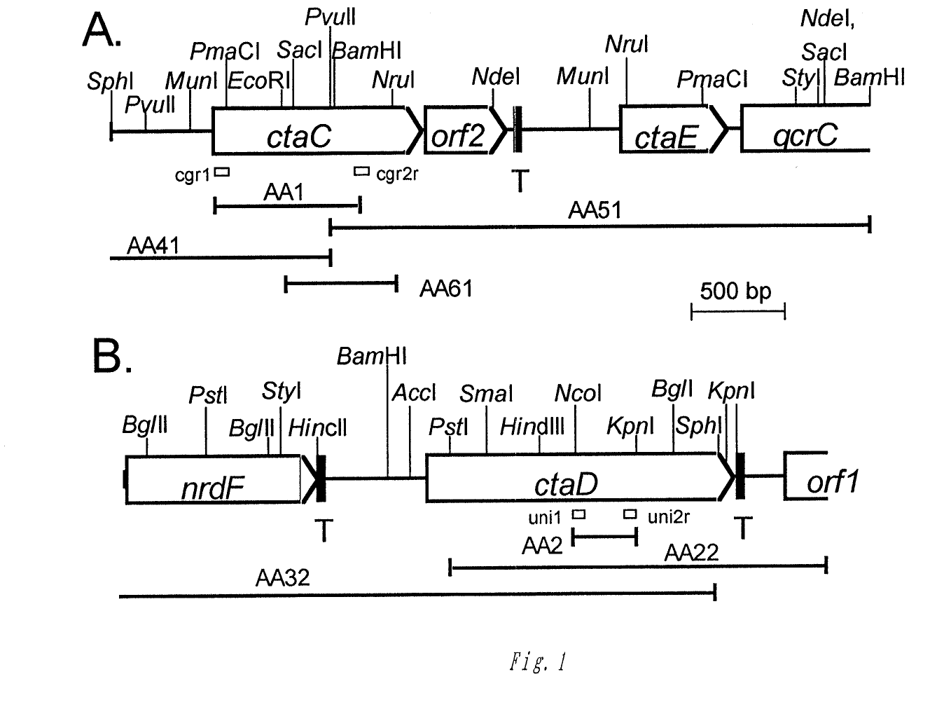

A labeled probe was produced in the same manner as the method described above by using the obtained fragment of 0.3 kb. By using this probe, Southern hybridization was performed for Corynebacterium glutamicum chromosomal DNA digested with PstI or SphI. DNA was extracted from the gel at a position corresponding to a position at which a positive signal was obtained. The DNA was ligated to pUC119 digested with PstI or SphI to prepare a recombinant DNA, and Escherichia coli was transformed with it. By using the aforementioned probe, colony hybridization was performed for colonies of transformants. Plasmids were prepared from colonies showing positive results for the hybridization to obtain clones AA22 and AA32. The nucleotide sequences of these clones were determined. The nucleotide sequence obtained by ligating the nucleotide sequences of these clones is shown in SEQ ID NO: 1. This nucleotide sequence contained two ORFs (FIG. 1B). As a result of database searching for these ORFs, it was found that the second ORF (nucleotide numbers 1538-3289) was the ctaD gene coding for the subunit I of aa3.

(2) Purification of Cytochrome aa3 of Corynebacterium glutamicum

Cultured bacterial cells (wet weight: 120 g) of Corynebacterium glutamicum KY9002 strain (ATCC13032) were suspended in 200 ml of a buffer (0.5% NaCl, 10 mM sodium phosphate, pH 7.4), and immediately disrupted by stirring at a high speed by means of a bead beater (Biospec) in the presence of glass beads (D=0.5 mm, 350 g). After this disrupted cell suspension was centrifuged at 8,000× g for 10 minutes to remove undisrupted bacterial cells, the supernatant was centrifuged at 100,000× g for 1 hour and the resulting precipitates were suspended in a buffer (100 mM NaCl, 50 mM potassium phosphate, pH 6.5) to obtain a membrane preparation.

The aforementioned membrane preparation was suspended in a buffer (5 mg/ml, 2% sodium cholate, 0.5 M NaCl, 10 mM sodium phosphate, pH 7.4), disrupted by sonication, and centrifuged at 100,000× g for 30 minutes. The resulting precipitates were suspended in a buffer (10 mg/ml, 2% n-octyl-D-glucoside, 0.5 M NaCl, 50 mM potassium phosphate, pH 6.5), and disrupted again by sonication. The disrupted suspension was centrifuged at 100,000× g for 30 minutes, and the obtained supernatant was dialyzed against a solution of 10 mM sodium phosphate, pH 7.4, and loaded on a DEAE-Toyopearl column (1.4×10 cm) so that proteinaceous components should be adsorbed on the column. The proteinaceous components were eluted with an eluent of 1% n-octyl-D-glucoside, 10 mM potassium phosphate, pH 7.4 with increasing NaCl concentration. A fraction showing spectra of heme a and heme a3 in the oxidation-reduction difference spectrum and CO binding reduction-type minus reduction-type difference spectrum was eluted with a condition of 200 mM NaCl as a single peak. This fraction was loaded on a hydroxyapatite column (0.8×2 cm) so that the proteinaceous components should be adsorbed on the column and eluted with an eluent of 1% n-octyl-D-glucoside containing sodium phosphate at a gradually increasing concentration. Since the fraction eluted with 200 mM sodium phosphate similarly showed spectra of heme a and heme a3 in the oxidation-reduction difference spectrum and CO binding reduction-type minus reduction-type difference spectrum and TMPD oxidase activity, it was concluded that it was a fraction containing cytochrome aa3 (Table 1).

TABLE 1

| | | | | TMPD oxidase activity | | |
|---|---|---|---|---|---|---|
| | | Cytochrome | | | Specific | |
| | Total | aa3 | | Total | activity | |
| | protein | | (nmol/) | activity | (unit/) | TN |
| | (mg) | (mmol) | mg) | (unit) | mg) | (s-1) |
| Membrane | 662 | 41.0 | 0.06 | 26.7 | 0.04 | 10.8 |
| Cholic acid | 583 | 42.7 | 0.07 | 19.4 | 0.033 | 7.51 |
| washing | | | | | | |
| Octylglucoside | 47.8 | 44.2 | 0.93 | 1.82 | 0.038 | 0.68 |
| extraction | | | | | | |
| DEAE-Toyopearl | 6.19 | 13.2 | 2.13 | 1.51 | 0.242 | 1.91 |
| Hydroxyapatite | 0.34 | 2.8 | 8.09 | 0.110 | 0.296 | 0.61 |

(3) Sequencing of N-terminus Amino Acid Sequence of Cytochrome aa3 Subunit II Protein

The cytochrome aa3 protein obtained in the manner described above was separated by SDS-PAGE and transferred to a polyvinylidene difluolide membrane. A portion of this membrane at a position considered to correspond to the subunit II was excised and used for amino acid sequencing in Pulse-liquid peptide sequencer Model 477A (Applied Biosystems). The determined amino acid sequence of the N-terminus portion is shown in SEQ ID NO: 13.

(4) Cloning of Cytochrome aa3 Subunit II and III Genes

A primer crg1 (GGYGAYTTCYTBCGNATGGG, SEQ ID NO: 14) was synthesized based on the amino acid sequence of the N-teminus amino acid sequence of the purified aa3 subunit II. Further, a primer crg2r (GGACCGCASARYTCNGMRCA, SEQ ID NO: 15) was synthesized based on the conserved sequence of CuA binding motif in the oxidase subunits II of Paracoccus denitrificans, Rhodobacter sphaeroides, Synechococcus vulcanus, Thermus thermophilus, Bacillus subtilis and Bacillus stearothermophilus. By using these crg1 and crg2r, and chromosomal DNA of Corynebacterium glutamicum KY9002 strain (ATCC13032) as a template, PCR was performed (95° C. for 60 seconds, 52° C. for 60 seconds, 68° C. for 60 seconds, 35 cycles) to obtain a fragment of 0.8 kb (AA1). The chromosomal DNA of Corynebacterium glutamicum was prepared by the method of Saito and Miura (Biochem. Biophys. Acta., 72, 619 (1963)).

The obtained fragment of 0.8 kb was labeled by using DIG DNA Labeling Kit (Bohringer Mannheim), and used as a probe for screening of a chromosomal DNA library. The used chromosomal DNA library of Corynebacterium glutamicum was obtained by partially digesting the chromosomal DNA with a restriction enzyme Sau3AI, ligating the obtained DNA fragments to pUC119 digested with a restriction enzyme BamHI to produce recombinant DNA, and transforming Escherichia coli with the recombinant DNA.

Colony hybridization (47-50° C., 5× SSC, 0.5% blocking reagent (Bohringer Mannheim), 0.1% sodium lauroyl sarcosinate, 0.02% SDS) was performed for the colonies of transformants by using the aforementioned probe labeled with DIG. The probe was detected by using DIG Detection Kit (Bohringer Mannheim), which utilized anti-DIG antibodies labeled with alkaline phosphatase. Plasmids were prepared from colonies that showed positive results for the hybridization to obtain clones AA41, AA51 and AA61. The nucleotide sequences of these clones were determined. It was found that these nucleotide sequences overlapped with on another and contained the ctaC gene coding for the subunit II and the ctaE gene coding for the subunit III of aa3 as a whole (FIG. 1A). A nucleotide sequence obtained by ligating the nucleotide sequences of the aforementioned three clones is shown in SEQ ID NO: 3. This nucleotide sequence contained three open reading frames (ORF). As a result of database searching for these ORFs, it was found that the first ORF (nucleotide numbers 604-1680) was the ctaC gene coding for the subunit II, and the third OFR (nucleotide numbers 2715-3329) was the ctaE gene coding for the subunit III. The amino acid sequences therefor are shown in SEQ ID NOS: 4 and 5, respectively.

The results of homology searching of databases for each amino acid sequence are shown in Table 2. The sequence analysis was performed by using GENETYX Homology Version 2.2.2 (Software Development Co., Ltd.). The homology analysis was performed according to the method of Lipman and Peason (Science, 227, 1435-1441, 1985).

TABLE 2

| | |
|---|---|
| Gene | Homology (amino acid sequence level) |

| | | |
|---|---|---|
| ctaD | Mycobacterium tuberculosis | ctaD product (70%) |
| | Bacillus stearothermohilus | Cytochrome oxidase |
| | | subunit I (47%) |
| ctaC | Mycobacterium tuberculosis | ctaC product (50%) |
| | Streptomyces coelicolor | Protein estimated to be |
| | | cytochrome oxidase |
| | | subunit II (32%) |
| ctaE | Mycobacterium tuberculosis | ctaE product (60%) |
| | Streptomyces coelicolor | Protein estimated to be |
| | | cytochrome oxidase |
| | | subunit III (57%) |

### EXAMPLE 2

### Cloning of Cytochrome bc1 Complex Gene

A sequence estimated to be a part of ORF was found in the sequence of SEQ ID NO: 3 obtained in Example 1 at a position downstream from the gene coding for cytochrome aa3 subunit III, ctaE, on the 3′ end side. As a result of database searching for the amino acid sequence that may be encoded by this sequence, the amino acid sequence showed homology to an amino acid sequence of known cytochrome c1 subunit.

The partial sequence of about 800 bp used for the cloning of ctaC in Example 1 was obtained by PCR, labeled by using DIG DNA Labeling Kit (Bohringer Mannheim), and designated as Probe AA1. By using this probe AA1, a chromosomal DNA library was screened through colony hybridization. The used chromosomal DNA library was obtained by partially digesting the chromosomal DNA of Corynebacterium glutamicum with a restriction enzyme Sau3AI, ligating the obtained DNA fragments to pUC18 digested with a restriction enzyme BamHI to produce recombinant DNA, and transforming Escherichia coli with the recombinant DNA. The probe was detected by using DIG Detection Kit (Bohringer Mannheim), which utilized anti-DIG antibodies labeled with alkaline phosphatase. Plasmids were prepared from colonies that showed positive results for the hybridization to obtain clone BLC1.

The nucleotide sequence of the aforementioned clone pBLC1 was determined, and a fragment of 154 bp was newly excised from that sequence with restriction enzymes EagI and SacI and designated as Probe 1. This Probe 1 was labeled in the same manner as described above, and used to screen again the chromosomal DNA library. The nucleotide sequence of clone B12 obtained by the above procedure was determined, and a fragment of about 500 bp excised from the sequence with a restriction enzyme StyI was used to produce a probe (Probe 2). This Probe 2 was labeled in the manner described above, and used to screen a chromosomal DNA library through colony hybridization. The chromosomal DNA library used in this screening was obtained by fully digesting the chromosomal DNA of Corynebacterium glutamicum with restriction enzymes BglII and SmaI, collecting DNA fragments of about 2.8 kb, ligating the fragment to pUC118 digested with restriction enzymes SmaI and BamHI to produce recombinant DNA, and transforming Escherichia coli with the recombinant DNA. This procedure provided a clone B13, and its nucleotide sequence was determined. A nucleotide sequence obtained by ligating BLC1, B12 and B13 is shown in SEQ ID NO: 6. This nucleotide sequence contained six ORFs, three of which (nucleotide numbers 276-1124, 1172-2347 and 2347-3963) were in an operon structure (FIG. 2). Databases were searched for these OFRs, and they were designated as qcrC, qcrA and qcrB, respectively. The amino acid sequences encoded by these ORFs are shown in SEQ ID NOS: 7, 8 and 10, respectively. In addition, the amino acid sequence encoded by qcrC and qcrA is shown in SEQ ID NO: 6 together with the nucleotide sequence. Further, the amino acid sequence encoded by qcrB is shown in SEQ ID NO: 9 together with the nucleotide sequence (the same the nucleotide sequence shown in SEQ ID NO: 6).

The qcrC gene product was characterized by containing two of heme c binding motifs, and showed 34% or more of homology on amino acid level to homologous proteins of high G+C content gram positive bacteria such as Mycobacterium tuberculosis and Streptomyces coelicolor. In particular, it showed a high homology of 54% to the homologous protein of M. tuberculosis. It was concluded that this qcrC was the gene coding for the cytochrome c1 subunit of cytochrome bc1 complex.

Further, the qcrA gene product contained a Rieske iron-sulfur motif, although the conservation degree was low, and showed the highest homology to the Rieske iron-sulfur protein of Thermus thermophilus among the high G+C content gram positive bacteria. It was concluded that this qcrA was the gene coding for Rieske iron-sulfur protein of the cytochrome bc1 complex.

The qcrB gene product showed homology to cytochrome b of high G+C content gram positive bacterium, T. thermophilus, and cytochrome b6 of Bacillus stearothermopholus, and a His residue that is a heme bonding site and peripheral sequences thereof were also highly conserved in it. It is expected that these gene products are functioning by forming a complex with cytochrome c1, iron-sulfur protein or cytochrome b, as is already known for mitochondria of eukaryotes and Proteobacteria. It was concluded that this qcrB was the gene coding for the cytochrome b subunit of cytochrome bc1 complex.

The sequence analysis was performed by using GENETYX Homology Version 2.2.2 (Software Development Co., Ltd.). The homology analysis was performed according to the method of Lipman and Peason (Science, 227, 1435-1441, 1985).

TABLE 3

| Gene | Homology (amino acid sequence level) |
|---|---|
| qcrC | qcrC product of high G + C content gram positive |
| | bacteria (34% or more) |
| | qcrC product of M. tuberculosis (54%) |
| qcrA | T. thermophilus Rieske iron-sulfur protein (45%) |
| qcrB | M. tuberculosis cytochrome b (61%) |
| | B. stearothermopholus cytochrome b6 (31%) |

### EXAMPLE 3

### Purification of Heme c Containing Protein of Corynebacterium glutamicum

(1) Purification of Heme c Containing Protein

In cytochrome c, heme c is covalently bonded to a cysteine residue of a protein portion, and therefore the heme c is still bonded to it even after the protein is denatured. The heme iron has peroxidase activity. Therefore, among polypeptides obtained by denaturing cytochrome c, presence of those having a covalently bonded heme can be clarified. Denatured cytochrome c was subjected to SDS-polyacrylamide gel electrophoresis, and peroxidase activity was detected. As a result, it was found that only the polypeptide having a molecular weight of about 28 kDa showed the peroxidase activity, and had c type heme. Based on this, it is considered that Corynebacterium glutamicum has one kind of protein containing heme c. Moreover, it is considered that, if this protein is purified and its amino acid sequence is determined, the subunit of cytochrome bc1 complex can be identified.

Cultured cells of Corynebacterium glutamicum KY9002 strain (ATCC13032) were collected by centrifugation at 10,000× g for 10 minutes, washed with a buffer (20 mM potassium phosphate, pH 7.5), and collected again by centrifugation at 10,000× g for 10 minutes. This washing procedure was repeated again, and the obtained cells were suspended in a buffer (20 mM potassium phosphate, pH 7.5), added with lysozyme (Sigma) at a final concentration of 0.5 mg/ml, left at room temperature for 30 minutes, and disrupted by sonication.

This disrupted cell suspension was centrifuged at 10,000× g for 20 minutes to remove undisrupted cells, and the supernatant was centrifuged at 150,000× g for 90 minutes to obtain precipitates, which were suspended in a buffer (20 mM potassium phosphate, pH 7.5) for washing. The suspension was centrifuged at 150,000× g for 90 minutes, and the obtained precipitates were suspended in 200 ml of buffer (1% sodium cholate, 0.5% sodium deoxycholate, 0.1 M NaCl, 10 mM sodium phosphate, pH 6.8) at a protein concentration of 10 mg/ml for washing, and the suspension was centrifuged at 100,000 x g for 45 minutes to precipitate membrane proteins.

The membrane proteins obtained as described above were dissolved in a buffer (1% decylglucoside, 0.05 M potassium phosphate, pH 6.5) by mild sonication, and centrifuged at 100,000× g for 30 minutes. The obtained supernatant was dialyzed against 20 mM Tris-HCl, pH 7.2 for 3 hours. The dialyzed supernatant was loaded on a DEAE-Toyopearl column (1.6×8.0 cm) equilibrated with a buffer (1% decylglucoside, 20 mM Tris-HCl, pH 7.2). The proteins adsorbed on the column were eluted with a buffer as eluent (1% n-octyl-D-glucoside, 20 mM Tris-HCl, pH 7.2) containing NaCl with increasing concentration. A fraction in red color containing heme c and exulted at 60-80 mM of NaCl concentration was collected, and loaded on a hydroxyapatite column (0.5×1.5 cm) so that the proteinaceous components should be adsorbed on the column. The column was washed with a buffer (1% n-octyl-D-glucoside, 20 mM Tris-HCl, pH 7.2), and then the proteinaceous components were eluted with a buffer (1% decylglucoside, 5 mM sodium phosphate, pH 6.8). The obtained fraction was concentrated to about 0.1 mL by loading it to Centricon 30 (Amersham), and subjected to gel filtration by loading it at a flow rate of 0.8 mL/min to a Toso G3000SW column (Tosoh) equilibrated with a buffer (0.05% decylglucoside, 0.1 M NaCl, 10 mM Tris-HCl, pH 7.2) by HPLC to obtain a fraction containing a protein containing heme c (Table 4).

TABLE 4

| | |
|---|---|
| Total protein | Heme c |

| | (mg) | (mmol) | (nmol/mg) |
|---|---|---|---|
| Membrane | 169 | 120.0 | 0.07 |
| Cholic acid washing | 166 | 99.0 | 0.06 |
| Octylglucoside | 4.5 | 33.0 | 7.3 |
| extraction | | | |
| DEAE-Toyopearl | 0.45 | 12.7 | 29 |
| Hydroxyapatite | 0.34 | 9.3 | 62 |

(2) Determination of N-terminus Amino Acid Sequence of Heme c Containing Protein

The fraction containing heme c containing protein obtained as described above was separated by SDS-PAGE and transferred to a polyvinylidene difluoride membrane. A portion of this membrane at a position corresponding to cc1 was excised, and the proteins were degraded composed by a previously reported method (Kuge, S. et al., Biochem. Mol. Biol. Int., 38, 181-188 (1996)) using Staphylococcus aureus V8 protease. The degradation product was subjected to amino acid sequence determination using Pulse-liquid peptide sequencer Model 477A (Applied Biosystems). As a result, two kinds of partial amino acid sequences, QAERKAPRITEAQVLA (SEQ ID NO: 16) and LRGENYDGQITSADVARGGDLFRL (SEQ ID NO: 17), were obtained. These partial amino acid sequences were substantially the same as the amino acid sequence obtained from the aforementioned cloned qcrC gene.

<Explanation of Sequence Listing>

SEQ ID NO: 1: nucleotide sequence of ctaD and amino acid sequence of CtaD,

SEQ ID NO: 2: amino acid sequence of CtaD,

SEQ ID NO: 3: nucleotide sequences of ctaC and ctaE and amino acid sequences of Cta C and CtaE

SEQ ID NO: 4: amino acid sequence of CtaC

SEQ ID NO: 5: amino acid sequence of CtaE

SEQ ID NO: 6: nucleotide sequence of qcrCAB and amino acid sequences of QcrC and QcrA

SEQ ID NO: 7: amino acid sequence of QcrC

SEQ ID NO: 8: amino acid sequence of QcrA

SEQ ID NO: 9: nucleotide sequence of qcrCAB and amino acid sequence of QcrB

SEQ ID NO: 10: amino acid sequence of QcrB

SEQ ID NO: 11: primer uni1

SEQ ID NO: 12: primer uni1r

SEQ ID NO: 13: N-terminus amino acid sequence of cytochrome aa3 subunit II protein

SEQ ID NO: 14: primer crg1

SEQ ID NO: 15: primer crg2r

SEQ ID NO: 16: partial amino acid sequence of cytochrome cc1 protein

SEQ ID NO: 17: partial amino acid sequence of cytochrome cc1 protein

What is claimed is:

1. A polypeptide defined in the following (A1) or (A2):
(A1) a polypeptide that has the amino acid sequence of SEQ ID NO: 2,
(A2) a polypeptide that has the amino acid sequence of SEQ ID NO: 2 comprising substitution, deletion, insertion or addition of one or several amino acid residues in the amino acid sequence, and can constitute cytochrome aa3 together with a cytochrome aa3 subunit II having the amino acid sequence of SEQ ID NO: 4 and cytochrome aa3 subunit III having the amino acid sequence of SEQ ID NO: 5.

2. A polypeptide defined in the following (B1) or (B2):
(B1) a polypeptide that has the amino acid sequence of SEQ ID NO: 4,
(B2) a polypeptide that has the amino acid sequence of SEQ ID NO: 4 comprising substitution, deletion, insertion or addition of one or several amino acid residues in the amino acid sequence, and can constitute cytochrome aa3 together with a cytochrome aa3 subunit I having the amino acid sequence of SEQ ID NO: 2 and cytochrome aa3 subunit III having the amino acid sequence of SEQ ID NO: 5.

3. A polypeptide defined in the following (C1) or (C2):
(C1) a polypeptide that has the amino acid sequence of SEQ ID NO: 5,
(C2) a polypeptide that has the amino acid sequence of SEQ ID NO: 5 comprising substitution, deletion, insertion or addition of one or several amino acid residues in the amino acid sequence, and can constitute cytochrome aa3 together with a cytochrome aa3 subunit I having the amino acid sequence of SEQ ID NO: 2 and cytochrome aa3 subunit II having the amino acid sequence of SEQ ID NO: 4.

4. A cytochrome aa3 consisting of the polypeptides according to claims 1, 2 and 3.

5. A polypeptide defined in the following (D1) or (D2):
(D1) a polypeptide that has the amino acid sequence of SEQ ID NO: 7,
(D2) a polypeptide that has the amino acid sequence of SEQ ID NO: 7 comprising substitution, deletion, insertion or addition of one or several amino acid residues in the amino acid sequence, and can constitute a cytochrome bc1 complex together with QcrA having the amino acid sequence of SEQ ID NO: 8 and QcrB having the amino acid sequence of SEQ ID NO: 10.

6. A polypeptide defined in the following (E1) or (E2):
(E1) a polypeptide that has the amino acid sequence of SEQ ID NO: 8,
(E2) a polypeptide that has the amino acid sequence of SEQ ID NO: 8 comprising substitution, deletion, insertion or addition of one or several amino acid residues in the amino acid sequence, and can constitute a cytochrome bc1 complex together with QcrC having the amino acid sequence of SEQ ID NO: 7 and QcrB having the amino acid sequence of SEQ ID NO: 10.

7. A polypeptide defined in the following (F1) or (F2):
(F1) a polypeptide that has the amino acid sequence of SEQ ID NO: 10,
(F2) a polypeptide that has the amino acid sequence of SEQ ID NO: 10 comprising substitution, deletion, insertion or addition of one or several amino acid residues in the amino acid sequence, and can constitute a cytochrome bc1 complex together with QcrC having the amino acid sequence of SEQ ID NO: 7 and QcrA having the amino acid sequence of SEQ ID NO: 8.

8. A cytochrome bc1 complex consisting of the polypeptides according to claims 5, 6 and 7.

9. A DNA coding for a polypeptide defined in the following (A1) or (A2):
(A1) a polypeptide that has the amino acid sequence of SEQ ID NO: 2,
(A2) a polypeptide that has the amino acid sequence of SEQ ID NO: 2 comprising substitution, deletion, insertion or addition of one or several amino acid residues in the amino acid sequence, and can constitute cytochrome aa3 together with a cytochrome aa3 subunit II having the amino acid sequence of SEQ ID NO: 4 and cytochrome aa3 subunit III having the amino acid sequence of SEQ ID NO: 5.

10. A DNA coding for a polypeptide defined in the following (B1) or (B2):
(B1) a polypeptide that has the amino acid sequence of SEQ ID NO: 4,
(B2) a polypeptide that has the amino acid sequence of SEQ ID NO: 4 comprising substitution, deletion, insertion or addition of one or several amino acid residues in the amino acid sequence, and can constitute cytochrome aa3 together with a cytochrome aa3 subunit I having the amino acid sequence of SEQ ID NO: 2 and cytochrome aa3 subunit III having the amino acid sequence of SEQ ID NO: 5.

11. A DNA coding for a polypeptide defined in the following (C1) or (C2):
(C1) a polypeptide that has the amino acid sequence of SEQ ID NO: 5,
(C2) a polypeptide that has the amino acid sequence of SEQ ID NO: 5 comprising substitution, deletion, insertion or addition of one or several amino acid residues in the amino acid sequence, and can constitute cytochrome aa3 together with a cytochrome aa3 subunit I having the amino acid sequence of SEQ ID NO: 2 and cytochrome aa3 subunit II having the amino acid sequence of SEQ ID NO: 4.

12. A DNA coding for a polypeptide defined in the following (D1) or (D2):
(D1) a polypeptide that has the amino acid sequence of SEQ ID NO: 7,
(D2) a polypeptide that has the amino acid sequence of SEQ ID NO: 7 comprising substitution, deletion, insertion or addition of one or several amino acid residues in the amino acid sequence, and can constitute a cytochrome bc1 complex together with QcrA having the amino acid sequence of SEQ ID NO: 8 and QcrB having the amino acid sequence of SEQ ID NO: 10.

13. A DNA coding for a polypeptide defined in the following (E1) or (E2):
(E1) a polypeptide that has the amino acid sequence of SEQ ID NO: 8,
(E2) a polypeptide that has the amino acid sequence of SEQ ID NO: 8 comprising substitution, deletion, insertion or addition of one or several amino acid residues in the amino acid sequence, and can constitute a cytochrome bc1 complex together with QcrC having the amino acid sequence of SEQ ID NO: 7 and QcrB having the amino acid sequence of SEQ ID NO: 10.

14. A DNA coding for a polypeptide defined in the following (F1) or (F2):
(F1) a polypeptide that has the amino acid sequence of SEQ ID NO: 10,
(F2) a polypeptide that has the amino acid sequence of SEQ ID NO: 10 comprising substitution, deletion, insertion or addition of one or several amino acid residues in the amino acid sequence, and can constitute a cytochrome bc1 complex together with QcrC having the amino acid sequence of SEQ ID NO: 7 and QcrA having the amino acid sequence of SEQ ID NO: 8.
